(19) United States
(12) Patent Application Publication
(10) Pub. No.: US 2012/0128755 A1
(43) Pub. Date: May 24, 2012

(54) Personal Care Composition Containing Yeast Extract And Hexapeptide

(76) Inventors: James Vincent Gruber, Washington, NJ (US); Dana Smith, Sickerville, NJ (US); Lisa Bouldin, Skillman, NJ (US)

(21) Appl. No.: 13/247,689

(22) Filed: Sep. 28, 2011

Related U.S. Application Data
(60) Provisional application No. 61/388,147, filed on Sep. 30, 2010.

(51) Int. Cl. A61K 9/127; A61Q 19/02; A61Q 17/04; A61P 31/00; A61P 29/00; A61K 8/99; A61K 36/06

(52) U.S. Cl. 424/450; 424/59; 424/62; 424/195.16

(57) ABSTRACT

A personal care composition comprising (a) a yeast extract, (b) Hexapeptide-11 (Phe-Val-Ala-Pro-Phe-Pro) (SEQ ID NO:1), and (c) a determatologically-acceptable carrier, wherein the total amount of components (a) and (b) is from about 2 wt % to about 6 wt %, preferably from 4-6% based on the total weight of the personal care composition. Also disclosed is a method for regulating skin conditions by using this composition.

### CROSS-REFERENCE TO RELATED APPLICATION

This application claims priority to U.S. provisional application No. 61/388,147, filed Sep. 30, 2010, which is herein incorporated by reference in its entirety.

### FIELD OF THE INVENTION

This invention relates generally to personal care compositions. More particularly, this invention relates to personal care compositions containing a blend of at least one yeast extract and at least one hexapeptide. The invention also relates to methods of using such personal care compositions.

### BACKGROUND OF THE INVENTION

Yeast extracts and hexapeptides are separately known and used in cosmetic compositions. Examples of prior references teaching the use of yeast extracts include the following: U.S. Pat. No. 2,230,479 discloses the topical use of yeast cell extracts to improve the oxygen uptake in human skin cells. U.S. Pat. No. 5,643,587 discloses that live yeast cell derivatives can find beneficial applications in compositions designed for under-eye skin lightening. U.S. Pat. No. 5,656,300 discloses that combinations of live yeast cell derivatives and minoxidil can improve the effectiveness of minoxidil treatments to grow human hair by synergistically stimulating the skin cells to help them respond to the minoxidil treatments. U.S. Pat. No. 5,676,956 teaches the use of live yeast cell derivatives to help reduce under-eye puffiness in humans. U.S. Pat. No. 5,676,973 teaches the use of live yeast cell derivatives in combination with fluorouracil to synergistically stimulate the effectiveness of fluorouracil to help in the treatment of skin acne. U.S. Pat. No. 6,177,105 teaches the use of live yeast cell derivatives to help stimulate the growth of collagen in human skin.

The use of stressed yeast lysates in cosmetic products is also known. Recently, it has been found that Saccharomyces cerevisiae, more commonly known as Baker's Yeast, can respond to growth stresses, such as heat shock, peroxides and ultraviolet light, to provide enhanced production of stress response agents. Illustratively, U.S. Pat. No. 5,776,441 discloses that UV stressed live yeast cell derivatives added to lip treatments can help to ameliorate the undesirable effects of dry and chapped lips. US Pat. Pub. No. 2006/0110815 teaches that ozone stressed yeast lysate provides skin cells with protection from exposure to ozone.

The use of hexapeptides in topical applications and cosmetics is also well established. For example, US Pat. Pub. No. 2008152606 discloses an acetylated hexapeptide of the structure Acetyl-Glu-Glu-Met-Glu-Arg-Arg to improve skin conditions associated with aging such as wrinkles, fine lines, laxity, mottled pigmentation, and sallowness. IE20060154 discloses that a hexapeptide of the structure Gly-Pro-Gln-Gly-Pro-Gln may improve the appearance of aging skin.

Likewise, peptides derived from yeast extracts, especially extracts from Saccharomyces cerevisiae (Bakers Yeast) can function topically to improve wound healing and the appearance of skin. See Bentley et al., Arch Surg 1990 (full citation). A peptide comprising the amino acid sequence Phe-Val-Ala-Pro-Phe-Pro (INCI name: Hexapeptide-11) was discovered in yeast ferments and was reported to firm aging skin. See Lupo et al., Dermatol Therapy 2007.

These references cited above, however, fail to examine the influence of combinations of yeast extracts with hexapeptides to synergistically influence skin. What is still needed is a personal care composition that exhibits benefits not noted from yeast extracts and hexapeptides when applied individually to skin or skin cells. This invention is believed to be an answer to the need.

### BRIEF SUMMARY OF THE INVENTION

Therefore, one aspect of the present invention is directed to a personal care composition containing: (a) a yeast extract, (b) Hexapeptide-11 (Phe-Val-Ala-Pro-Phe-Pro) (SEQ ID NO:1), and (c) a determatologically-acceptable carrier, wherein the total amount of components (a) and (b) is from about 2 wt % to about 6 wt %, preferably from about 4 wt % to about 6 wt % based on the total weight of the personal care composition.

Another aspect of the present invention is directed to a method for regulating skin condition comprising topically applying to skin a personal care composition containing: (a) a yeast extract, (b) Hexapeptide-11 (Phe-Val-Ala-Pro-Phe-Pro) (SEQ ID NO:1), and (c) a determatologically-acceptable carrier, wherein the total amount of components (a) and (b) is from about 2-6%, preferably from 4-6% based on the total weight of the personal care composition.

These and other aspects will become apparent upon reading the following detailed description of the invention.

### BRIEF DESCRIPTION OF THE DRAWINGS

FIG. 1 illustrates the in vitro effect of yeast extract and yeast-derived hexapeptide on type 1A1 collagen expression.

### DETAILED DESCRIPTION OF THE INVENTION

The Personal care composition of the invention can comprise a yeast extract, a hexapeptide, preferably Hexapeptide-11 (Phe-Val-Ala-Pro-Phe-Pro) (SEQ ID NO:1), and a determatologically-acceptable carrier, as well as any of the additional or optional ingredients, components, or limitations described herein. Preferably, the yeast extract and the hexapeptide are present in an amount of from about 2 wt % to about 6 wt %, more preferably from about 4 wt % to 6 wt %, and the carrier is present in an amount of from about 50 wt % to about 99 wt %, all based on the total weight of the personal care composition. In one embodiment, the weight ratio of the yeast extract to the hexapeptide is between about 1:4 to about 4:1, preferably between about 1:2 to about 2:1. In one embodiment, the yeast extract and the Hexapeptide-11 are encapsulated in a delivery vehicle such as liposome, niasome, nanosome.

As used herein, personal care compositions encompass a wide variety of applications, including but not limited to soaps, shampoos, skin care medicaments, cosmetics, therapeutic and homeopathic skin care formulations.

The personal care compositions of the present invention, including the essential and some optional components thereof, are described in detail hereinafter.

### Yeast Extract

The yeast extract of the present invention is obtained through standard fermentation processes known to those skilled in the art. As defined for the purposes of this invention, a yeast extract is a composition derived from yeast grown on a nutritional growth media that is subsequently killed in such a way as to afford a product that includes cellular yeast components including, but not limited to, the nutrient broth, cellular protein material, cellular nuclear material, cellular cytoplasmic material, cellular protoplasmic material and/or cell wall components. Typically, the yeast extract is essentially water-soluble. For purposes of this disclosure, water-soluble means 0.1 gram of yeast components are dissolved in 1 gram of water.

The yeast used in the fermentation process can be of various genus known to those skilled in the art including, but not limited to: Arthroascus, Aureobasidium, Botryoascus, Brettanomyces, Candida, Citeromyces, Clavispora, Cryptococcus, Debaryomyces, Dekkera, Filobasidium, Guilliermondella, Hansenula, Haneseniaspora, Hormoascus, Klockera, Kluyveromyces, Leucosporidium, Lipomyces, Malassezia, Metschnikowia, Nadsonia, Nematospora, Oosporidium, Pachysolen, Pachytichospora, Penicillium, Pichia, Prototheca, Rhodosporidium, Rhodotorula, Saccharomyces, Saccharomycodes, Saccharomycopsis, Schizosaccharomyce, Schwanniomyces, Sporobolomyces, Sporopachydermia, Tremella, Trichosporan, Trigonopsis, Torulaspora, Torulopsis, Williopsis, Yarrowia, Zygosaccharomyces. In one embodiment, suitable yeasts are those based on the genus Saccharomyces, in particular Saccharomyces cerevisiae, more commonly known as Baker's Yeast.

The yeast growth media employed in the present invention, frequently called a growth peptone, can be comprised of a variety of ingredients as might be found, for example, in Atlas, RM “Handbook of Microbiological Media” ed., Parks, LC, CRC Press, Boca Raton, Fla., 1993. In particular, the media might be similar to the media found on page 1006 of the above text defined as “Yeast Fermentation Medium”. Such media are commercially available from, for example, Sigma Life Sciences, St. Louis, Mo.

Methods for growing the yeast of the present invention are known to those skilled in the arts. Generally speaking, the yeast can be grown simply in an open air fermentation vessel, or under more sophisticated conditions as might be done using a sealed biological fermentor as might be available from New Brunswick Scientific, Edison, N.J.

After the fermentation, the yeast is then lysed to obtain yeast extract. The yeast can be lysed by a variety of methods known to one skilled in the art, including but not limited to, enzymes, high-speed agitation, changes in growth media, autolysis or changes in pH. The yeast lysate typically contains water-soluble and water-insoluble components. The water-insoluble components may be separated from the water-soluble components. If desired, the yeast extract may be further purified via methods known to a person skilled in the art.

### Hexapeptide

An essential component of the present invention is a peptide isolated either through biological means such as fermentation or via more classic methods such as solid state or solution phase synthetic chemistry. More particularly, of importance to the present invention are peptides comprising essentially six amino acids, known collectively as hexapeptides. The amino acids of the hexapeptide can be any of the naturally-occurring amino acids or it may comprise amino acids formed through unnatural synthetic processes.

Of particular interest is a hexapeptide isolated from yeast ferments known as Hexapeptide-11 (chemical structure: Phe-Val-Ala-Pro-Phe-Pro) (SEQ ID NO:1) [Lupo et al., Dermatol Therapy 2007]. The structure of Hexapeptide-11 is shown schematically below:

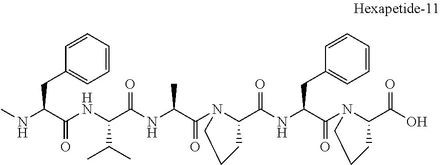

The Hexapeptide of the present invention can be derived from yeasts or can be provided as a synthetic peptide made through standard methods known to those skilled in the art. The hexapetide of the present invention can be further derivatized such as, for example, esterified or converted into an amide through standard synthetic processes known to those skilled in the art.

### Carrier

Another essential ingredient of the present invention is a dermatologically acceptable carrier. The term “dermatologically-acceptable,” as used herein, means that the compositions or components thereof so described are suitable for use in contact with human skin without undue toxicity, incompatibility, instability, allergic response, and the like.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse. Other suitable topical carriers include anhydrous liquid solvents such as oils, alcohols, and silicones (e.g., mineral oil, ethanol, isopropanol, dimethicone, cyclomethicone, and the like); aqueous-based single phase liquid solvents (e.g., hydro-alcoholic solvent systems); and thickened versions of these anhydrous and aqueous-based single phase solvents (e.g., where the viscosity of the solvent has been increased to form a solid or semi-solid by the addition of appropriate gums, resins, waxes, polymers, salts, and the like). Examples of topical carrier systems useful in the present invention are described in the following four references all of which are incorporated herein by reference in their entirety: “Sun Products Formulary” Cosmetics & Toiletries, vol. 105, pp. 122-139 (December 1990); “Sun Products Formulary”, Cosmetics & Toiletries, vol. 102, pp. 117-136 (March 1987); U.S. Pat. No. 4,960,764 to Figueroa et al., issued Oct. 2, 1990; and U.S. Pat. No. 4,254,105 to Fukuda et al., issued Mar. 3, 1981.

The carriers of the present invention can comprise from about 50% to about 99% by weight of the compositions of the present invention, preferably from about 75% to about 99%, and most preferably from about 85% to about 95%.

Preferred dermatologically acceptable carrier include hydro-alcoholic systems and oil-in-water emulsions. When the carrier is a hydro-alcoholic system, the carrier can comprise from about 0% to about 99% of ethanol, isopropanol, or mixtures thereof, and from about 1% to about 99% of water. More preferred is a carrier comprising from about 5% to about 60% of ethanol, isopropanol, or mixtures thereof, and from about 40% to about 95% of water. Especially preferred is a carrier comprising from about 20% to about 50% of ethanol, isopropanol, or mixtures thereof, and from about 50% to about 80% of water. When the carrier is an oil-in-water emulsion, the carrier can include any of the common excipient ingredients for preparing these emulsions. A more detailed discussion of suitable carriers is found in U.S. Pat. No. 5,605,894 to Blank et al., and, U.S. Pat. No. 5,681,852 to Bissett, both of which are herein incorporated by reference in their entirety.

### Optional Components

The compositions of the present invention may optionally comprise additional skin actives. Non-limiting examples of such skin actives include vitamin B3 compounds such as those described in PCT application WO 97/39733, published Oct. 30, 1997, to Oblong et al., hydroxy acids such as salicylic acid; exfoliation or desquamatory agents such as zwitterionic surfactants; sunscreens such as 2-ethylhexyl-p-methoxycinnamate, 4,4′-t-butyl methoxydibenzoyl-methane, octocrylene, phenyl benzimidazole sulfonic acid; sun-blocks such as zinc oxide and titanium dioxide; anti-inflammatory agents; anti-oxidants/radical scavengers such as tocopherol and esters thereof; metal chelators, especially iron chelators; retinoids such as retinol, retinyl palmitate, retinyl acetate, retinyl propionate, and retinal; N-acetyl-L-cysteine and derivatives thereof; hydroxy acids such as glycolic acid; keto acids such as pyruvic acid; benzofuran derivatives; depilatory agents (e.g., sulfhydryl compounds); skin lightening agents (e.g., arbutin, kojic acid, hydroquinone, ascorbic acid and derivatives such as ascorbyl phosphate salts, placental extract, and the like); anti-cellulite agents (e.g., caffeine, theophylline); moisturizing agents; anti-microbial agents; anti-androgens; and skin protectants. Mixtures of any of the above mentioned skin actives may also be used. A more detailed description of these actives is found in U.S. Pat. No. 5,605,894 to Blank et al. Preferred skin actives include hydroxy acids such as salicylic acid, sunscreen, antioxidants and mixtures thereof.

Other conventional skin care product additives may also be included in the compositions of the present invention. For example, urea, guanidine, glycerol, petrolatum, mineral oil, sugar esters and polyesters, polyolefins, methyl isostearate, ethyl isostearate, cetyl ricinoleate, isononyl isononanoate, isohexadecane, lanolin, lanolin esters, cholesterol, pyrrolidone carboxylic acid/salt (PCA), trimethyl glycine (betaine), tranexamic acid, amino acids (e.g., serine, alanine, threonine, histidine) and/or their salts, panthenol and its derivatives, collagen, hyaluronic acid, elastin, hydrolysates, primrose oil, jojoba oil, epidermal growth factor, soybean saponins, mucopolysaccharides, and mixtures thereof may be used. Other suitable additives or skin actives are discussed in further detail in PCT application WO 97/39733, published Oct. 30, 1997, to Oblong et al.

The formulation also can comprise other components that may be chosen depending on the carrier, optional components or the intended use of the formulation. Additional components include, but are not limited to antioxidants (such as BHT); emulsion stabilizers (such as carbomer); preservatives (such as phenoxyethanol); fragrances (such as pinene); humectants (such as glycerine); waterproofing agents (such as Fomblins perflouorethers); water-soluble film formers (such as hydroxypropyl methylecellulose); oil-soluble film formers (such as hydrogenated C-9 resins); moisturizing agents (such as cholesterol); cationic polymers (such as Polyquaternium-10); anionic polymers (such as xanthan gum); vitamins (such as tocopherol); and the like.

Particularly preferred embodiments of the present formulations are skin care lotions or creams used as anti-aging products. To that end, the present formulations are combined with agents that are moisturizers, emollients or humectants. Examples of useful combinations are oils, fats, waxes, esters, fatty acid alcohols, fatty acid ethoxylates, glycols, sugars, hyaluronic acid and hyaluronates, dimethicone, cyclomethicone, and the like. Further examples can be found in the International Cosmetic Ingredient Dictionary CTFA, Tenth Edition, 2004.

### Preparation of Compositions

The compositions of the present invention are generally prepared by conventional methods such as are known in the art of making topical compositions. Such methods typically involve mixing of the ingredients in one or more steps to a relatively uniform state, with or without heating, cooling, application of vacuum, and the like.

### Methods for Regulating Skin Condition

The personal care compositions of the invention are useful for topical application and for regulating skin condition, such as, regulating visible and/or tactile discontinuities in the texture of skin, reducing post-inflammatory hyperpigmentation, regulating non-melanin discoloration of skin, regulating moisturization and barrier properties of skin, regulating epidermal differentiation of skin, regulating exfoliation of skin, thickening of skin to reduce skin atrophy, regulating the elasticity of skin, reducing oily skin, regulating cellulite in skin, regulating pruritus in skin, promoting wound healing in skin and regulating nerve growth and nerve function in the skin. Regulating skin condition normally involves improving skin appearance and/or feel.

Regulating skin condition involves topically applying to the skin a safe and effective amount of a composition of the present invention. The amount of the composition which is applied, the frequency of application and the period of use will vary widely depending upon the level of the yeast and hexapeptide and/or other components of a given composition and the level of regulation desired.

In a preferred embodiment, the composition is chronically applied to the skin. By “chronic topical application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. Typically applications would be on the order of about once per day over such extended periods, however application rates can vary from about once per week up to about three times per day or more.

In one embodiment, regulating skin condition is practiced by applying the personal care composition of the invention in the form of a skin lotion, cream, gel, emulsion, spray, conditioner, cosmetic, lipstick, foundation, nail polish, or the like to any part of the external portion of the face, hair, and/or nails can be treated, e.g., face, lips, under-eye area, eyelids, scalp, neck, torso, arms, hands, legs, fingernails, toenails, scalp hair, eyelashes, eyebrows, etc.

One approach to ensure a continuous exposure of the skin to at least a minimum level of the personal care composition of the present invention is to apply the composition by use of a patch applied, e.g., to the face. Such an approach is particularly useful for problem skin areas needing more intensive treatment. The patch can be occlusive, semi-occlusive or non-occlusive. The personal care composition can be contained within the patch or be applied to the skin prior to application of the patch. The patch can also include additional actives such as chemical initiators for exothermic reactions such as those described in PCT application WO 9701313 to Burkett et al.

Another approach for applying the composition of the present invention is through a rinse-off composition such as, but not limited to, a shampoo, conditioner, body wash, facial scrub, facial peel and the like.

It is believed that yeast extract and hexapeptide-11 of the present invention synergistically stimulate extracellular matrix protein production in skin cells, such as fibroblasts. In one embodiment, the composition of the present invention is effective in increasing expression of Type 1A1 collagen in normal human dermal fibroblasts.

The following examples are illustrative and not to be construed as limiting of the invention as disclosed and claimed herein. All parts and percentages are by weight and all temperatures are degrees Celsius unless explicitly stated otherwise. All patent applications, patents and other publications cited herein are incorporated by reference in their entirety.

### Example 1

### Isolation of Yeast Extract Grown from Saccharomyces Organism and Media

S. cerevisiae (Red Star baker's yeast) was used to prepare the yeast extract of the present invention. Stock culture was maintained on yeast peptide dextrose (YPD) agar slant (Difco). The working culture was maintained in YPD broth at 4° C. The fermentation was carried out with the medium containing 10 g/L yeast extract, 8 g/L NH₄SO₄, 3 g/L KH₂PO₄, 2 g/L MgSO₄, and 0.5 mL/L Antifoam A. Unless otherwise stated the working volume for fermentation was 2 L.

### Bioreactor

A New Brunswick Bioflo 110 benchtop bioreactor (Edison, N.J.) equipped with automatic pH, temperature, agitation, dissolved oxygen (DO) and antifoam controls was used. The 2-L vessel was equipped with air in- and out-ports, alkali and medium addition ports, and effluent side ports. Medium pH was maintained at 5.5 by adding 4 M NaOH and/or 4 M H₂SO₄. Aeration was maintained at 1 vvm (volume of air/working volume of fermentor/min) and DO level was kept at 60% by cascading to the agitation. The fermentor was fed with 250 g/L of glucose solution at the rate of 1.2 mL/h.

### Preparation of Yeast Extract

Samples of S. cerevisiae for analytical tests were taken from fermentation broth after 48 h at 1.0×10⁹ cells/mL. Forty five mL of culture sample was centrifuged (15000 rpm for 10 min) and washed with distilled water. The precipitate was resuspended in 45 mL of 10 mM phosphate buffer, pH 7, supplemented with phenylmethylsulphonyl fluoride (Sigma Chemical Co., St. Louis, Mo.) to 1 mM and pepstatin A (Sigma) to a concentration of 10 μM. Forty five mL glass beads (0.5 mm diameter; Biospec Products, USA) was added to the cell suspension and the suspension in 90 mL container was inserted into a Beadbeater (Biospec Products) and shaken at homogenization speed for 3 min. The sample chamber was cooled during the homogenization step by ice bath. The protein concentration of samples was determined by BCA protein assay (Sigma). Samples of each lysate were analyzed as described in Example 2.

### Example 2

### Preparation of Yeast-Derived Hexapeptide

Yeast (Saccharomyces cerevisiae) was grown according to the conditions outlined in Jazwinski S M. Methods in Enzymology 182 (1990)154-174 incorporated in its entirety. Upon completion of the fermentation process, the yeast was isolated by filtration and resuspended in PBS. The microorganisms were ruptured by running the mixture through a microfluidizer to provide a mixture of ruptured yeast cells and cytoplasmic contents. The undissolved components, which included principally cell wall components, were removed by filtration to provide a mixture of water-soluble materials containing peptides, oligopeptides, sugars and polymeric sugars among other components.

The resulting yeast extract was first fractionated for molecular weight distribution using tangential flow filtration employing a membrane filter of nominal molecular weight cut-off at 3000 daltons. The resulting low molecular weight fraction was further fractionated using High Performance Liquid Chromatography using the following conditions: Column: C18 (1.0×250 mm), Mobile Phase: 5% to 80% of a mixture of 0.1% trifluoroacetic acid in water) and 0.0075% trifluoroacetic acid in 70% acetonitrile. Fractions taken from the chromatography column were isolated and the component of the largest fraction was concentrated to provide a fraction containing the hexapeptide of the present invention, namely, Phe-Val-Ala-Pro-Phe-Pro (SEQ ID NO:1), the structure being identified via Edman Degradation to determine the amino acid sequence.

### Example 3

### In Vitro Effect of Yeast Extract and Yeast-Derived Peptide on Type 1A1 Collagen Expression

### Preparation of Fibroblasts

Fibroblasts were seeded into the individual wells of a 24-well plate in 0.5 ml of Fibroblast Growth Media (FGM) and incubated overnight at 37±2° C. and 5±1% CO₂. On the following day the media was removed via aspiration to eliminate any non-adherent cells and replaced with 0.5 ml of fresh FGM. The cells were grown until confluent, with a media change every 48 to 72 hours. Upon reaching confluency the cells were treated for 24 hours with DMEM supplemented with 1.5% FBS to wash out any effects from the growth factors included in the normal culture media. After this 24-hour wash out period the cells were treated with the test materials at the specified concentrations dissolved in DMEM with 1.5% FBS. Untreated cells (negative controls) just received DMEM with 1.5% FBS. The cells were incubated for 48 hours and at the end of the incubation period cell culture medium was collected and either stored frozen (−75° C.) or assayed immediately. Materials were tested in triplicate.

### Procollagen Assay

A series of type I C-peptide standards was prepared ranging from 0 ng/ml to 640 ng/ml. Next, an ELISA microplate was prepared by removing any unneeded strips from the plate frame followed by the addition of 100 μl of peroxidase-labeled anti procollagen type I-C peptide antibody to each well used in the assay. Twenty (20) μl of either sample (collected tissue culture media) or standard was then added to appropriate wells and the microplate was covered and allowed to incubate for 3±0.25 hours at 37° C. After the incubation the wells were aspirated and washed three times with 400 μl of wash buffer. After the last wash was removed 100 μl of peroxidase substrate solution (hydrogen peroxide+tetramethylbenzidine as a chromagen) was added to each well and the plate was incubated for 15±5 minutes at room temperature. After the incubation 100 μl of stop solution (1 N sulfuric acid) was added to each well and the plate was read using a microplate reader at 450 nm.

### Procollagen ELISA

To quantify the amount of each procollagen present, a standard curve was generated using known concentrations of procollagen type I C-peptide. A regression analysis was performed to establish the line that best fits these data points. Absorbance values for the test materials and untreated samples were used to estimate the amount of procollagen type I C-peptide present in each sample.

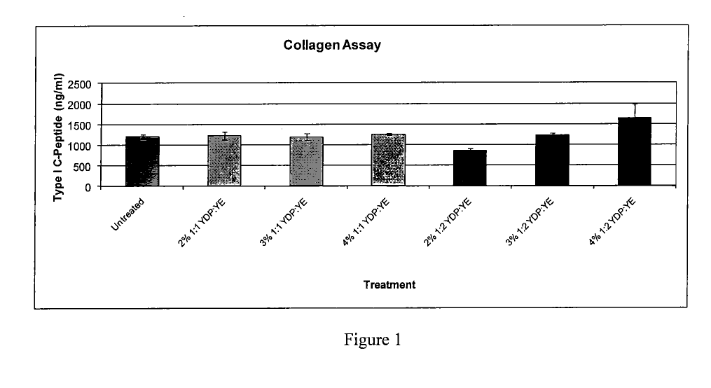

Data from these Studies is Shown in FIG. 1.

The data demonstrates that at a 4% concentration the combination of 1 Part Yeast Peptide (YDP) and 1 Part Yeast Extract (YE) showed statistically significant improvement in collagen expression compared to the untreated control.

### Example 4

### Encapsulation of the Yeast Extract and Hexapeptide Blend in Liposome

Samples of the yeast extract and yeast-derived peptide prepared as shown in Examples 1 and 2 above were incorporated into a liposome comprising phospholipid and lecithin obtained from soybean beans. The synergistic blend was slurried together with the phospholipid and lecithin components and the mixture was homogenized using a high-pressure homogenizer obtained from Hydraulic Engineering Corporation (Brea, Calif.). The milky white mixture contained the synergistic blend of yeast extract and yeast-derived peptide was encapsulated with the liposomal components.

### Example 5

### Encapsulation of the Yeast Extract and Hexapeptide Blend in a Maltodextrin

Samples of the yeast extract and yeast-derived peptide prepared as shown in Examples 1 and 2 above are incorporated into a maltodextrin according to the procedures shown in example 4.

While the invention has been described above with references to specific embodiments thereof, it is apparent that many changes, modifications and variations can be made without departing from the inventive concept disclosed herein. Accordingly, it is intended to embrace all such changes, modifications and variations that fall within the spirit and broad scope of the appended claims.

What is claimed is:

1. A personal care composition comprising (a) a yeast extract, (b) Hexapeptide-11 (Phe-Val-Ala-Pro-Phe-Pro) (SEQ ID NO:1), and (c) a determatologically-acceptable carrier, wherein the total amount of components (a) and (b) is from about 2 wt % to about 6 wt % based on the total weight of the personal care composition.

2. The composition of claim 1 wherein the total amount of components (a) and (b) is from about 4% to about 6% based on the total weight of the personal care composition.

3. The composition of claim 1 wherein the weight ratio of component (a) relative to component (b) is between about 1:4 to about 4:1.

4. The composition of claim 3 wherein the weight ratio of component (a) relative to component (b) is between about 1:2 to about 2:1.

5. The composition of claim 1 wherein the yeast extract is derived from Saccharomyces cerevisiae.

6. The composition of claim 1 wherein the carrier is an emulsion selected from the group consisting of water-in-oil, oil-in-water, water-in-oil-in-water, and oil-in-water-in-silicone emulsions.

7. The composition of claim 1 wherein the carrier is selected from the group consisting of water, oil, alcohol, silicone, and combinations thereof.

8. The composition of claim 1 wherein components (a) and (b) are encapsulated in a delivery vehicle selected from the group consisting of liposome, niasome, nanosome, and combinations thereof.

9. The composition of claim 1 further comprising at least one ingredient selected from the group consisting of hydroxyl acids, exfoliation or desquamatory agents, sunscreens, sun-blocks, anti-inflammatory agents, anti-oxidants/radical scanvengers, metal chelators, keto acids, depilatory agents, skin lightening agents, anti-cellulite agents, moisturizing agents, anti-microbial agents; anti-androgens, skin protectants, emulsion stabilizers, preservatives, fragrances, humectants, waterproofing agents, water-soluble film formers, oil-soluble film formers, cationic polymers, vitamins, and combinations thereof.

10. The composition of claim 1 wherein component (a) and component (b) synergistically stimulate extracellular matrix protein production in skin cells.

11. The composition of claim 10 wherein the skin cells are fibroblasts.

12. The composition of claim 1 wherein the composition is effective in increasing expression of Type 1A1 collagen in normal human dermal fibroblasts.

13. A method for regulating skin conditions comprising contacting the skin with a personal care composition containing (a) a yeast extract, (b) Hexapeptide-11 (Phe-Val-Ala-Pro-Phe-Pro) (SEQ ID NO:1), and (c) a determatologically-acceptable carrier, wherein the total amount of components (a) and (b) is from about 2 wt % to about 6 wt % based on the total weight of the personal care composition.

14. The method of claim 13 wherein the total amount of components (a) and (b) is from about 4% to about 6% based on the total weight of the personal care composition.

15. The method of claim 13 wherein the weight ratio of component (a) relative to component (b) is between about 1:4 and about 4:1.

16. The method of claim 13 wherein the weight ratio of component (a) relative to component (b) is between about 1:2 and about 2:1.

17. The method of claim 13 wherein the carrier is an emulsion selected from the group consisting of water-in-oil, oil-in-water, water-in-oil-in-water, and oil-in-water-in-silicone emulsions.


SEQUENCE LISTING (1 sequence)

SEQ ID NO: 1 - protein, 6 aa, Saccharomyces cerevisiae
FVAPFP